(19) United States
(12) Patent Application Publication
(10) Pub. No.: US 2024/0358528 A1
(43) Pub. Date: Oct. 31, 2024

(54) FINGER PROSTHETIC

(71) Applicant: Intermed Labs @ Mon Health, LLC, Morgantown, WV (US)

(72) Inventors: William T. MCCLELLAN, Morgantown, WV (US); Justin R. CHAMBERS, Morgantown, WV (US); Ephraim PITTORE-CANDELARIO, Morgantown, WV (US); Zachary HOOPES, Morgantown, WV (US)

(21) Appl. No.: 18/765,730

(22) Filed: Jul. 8, 2024

Related U.S. Application Data
(62) Division of application No. 17/890,587, filed on Aug. 18, 2022.
(60) Provisional application No. 63/234,431, filed on Aug. 18, 2021.

(51) Int. Cl. A61F 2/58; A61F 2/50

(52) U.S. Cl. CPC A61F 2/586; A61F 2/5046

(57) ABSTRACT

A prosthetic finger device includes a base piece configured to fit on a proximal phalanx portion of the amputated finger, and a tip piece configured to fit on a middle phalanx portion of the amputated finger, wherein the tip piece is pivotally connected to the base piece by a hinge that is integrally formed with the tip piece and the base piece.

### PRIORITY

This application is a divisional of application Ser. No. 17/890,587 filed Aug. 18, 2022, which claims priority to provisional application No. 63/234,431 filed Aug. 18, 2021, the disclosures of which are incorporated by reference herein in their entirety.

### FIELD OF THE INVENTION

The invention generally relates to medical devices and associated methods of manufacture and use, and more particularly to prosthetics for fingers.

### BACKGROUND

There are about 50,000 new finger amputations that occur yearly in the United States, and over 1 million people without some part of a finger (also referred to as a digit). Prosthetic use for missing parts of fingers can be limited due to access and cost.

FIG. 1 shows the bones of a finger including the proximal phalanx (P1), middle phalanx (P2), and distal phalanx (P3). One type of finger amputation results in the patient losing the distal phalanx (P3) while maintaining the proximal phalanx (P1) and the middle phalanx (P2). Another type of finger amputation results in the patient losing the distal phalanx (P3) and the middle phalanx (P2), while maintaining the proximal phalanx (P1).

### SUMMARY

Implementations of the invention address the above-noted problems of the prior art by providing a prosthetic designed to replace missing fingertips due to a loss or amputation of the distal phalanx (P3) or both the distal phalanx (P3) and the middle phalanx (P2). In embodiments, the prosthetic is created by digitally scanning the hand, digitally generating a custom device model, and digitally manufacturing the prosthetic to be shipped directly to the patient.

Implementations of the invention provide an effective and low-cost alternative to the high-end/expensive prosthetics, while still maintaining comparable function. Implementations of the prosthetics described herein benefit the patients by helping with pinching objects, typing, and other fine motor skills.

According to aspects of the invention, the prosthetic sizing is achieved by digitally scanning with a smart device to size the customers fingers via artificial intelligence (AI) measuring systems. In embodiments, this system uses photographs of the customer's hand to calculate the dimensions for the prosthetic to fit without the need for in person consultations.

In a first aspect of the invention, there is a prosthetic device for use with an amputated finger that is missing some or all of a distal phalanx, the device consisting of: a base piece configured to fit on a proximal phalanx portion of the amputated finger; and a tip piece configured to fit on a middle phalanx portion of the amputated finger, wherein the tip piece is pivotally connected to the base piece by a hinge that is integrally formed with the tip piece and the base piece.

In another aspect of the invention, there is a prosthetic device for use with an amputated finger that is missing a distal phalanx and some or all of a middle phalanx, the device comprising: a base piece configured to fit on a proximal phalanx portion of the amputated finger; a tip piece pivotally connected to the base piece by a hinge that is integrally formed with the tip piece and the base piece; a base ring configured to fit on a proximal phalanx portion of a finger adjacent to the amputated finger, wherein the base ring is connected to the base piece; and a tip ring configured to fit on a middle phalanx portion of the finger adjacent to the amputated finger, wherein the tip ring is connected to the tip piece.

In another aspect of the invention, there is a method of manufacturing a prosthetic device for use with an amputated finger, the method comprising: receiving at least one digital image that includes an image of the amputated finger and an object of known size; determining at least one dimension of the amputated finger using the at least one digital image; generating a CAD file for the prosthetic device based on the determined at least one dimension; and digitally manufacturing the prosthetic device using the CAD file.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention.

FIG. 1 shows the bones of a finger.

FIGS. 2A and 2B show a finger prosthetic in accordance with aspects of the invention.

FIGS. 3A and 3B show a finger prosthetic in accordance with aspects of the invention.

FIG. 4 shows a finger prosthetic installed on a patient hand in accordance with aspects of the invention.

FIGS. 5A and 5B show a finger prosthetic in accordance with aspects of the invention.

FIGS. 6A and 6B show a finger prosthetic relative to and installed on a patient hand in accordance with aspects of the invention.

FIG. 7 shows a finger prosthetic in accordance with aspects of the invention.

FIGS. 8A, 8B, and 8C show a finger prosthetic in accordance with aspects of the invention.

FIG. 9 shows a flowchart of a method in accordance with aspects of the invention.

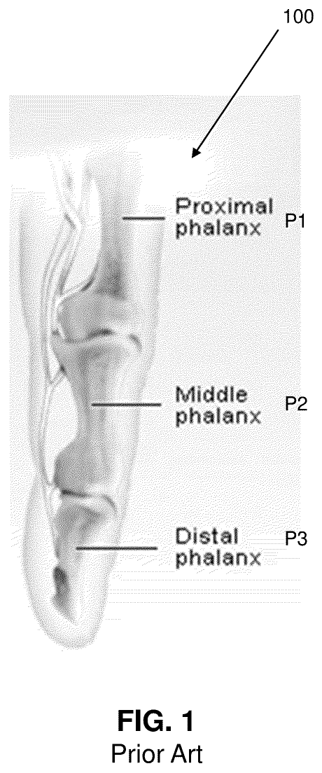

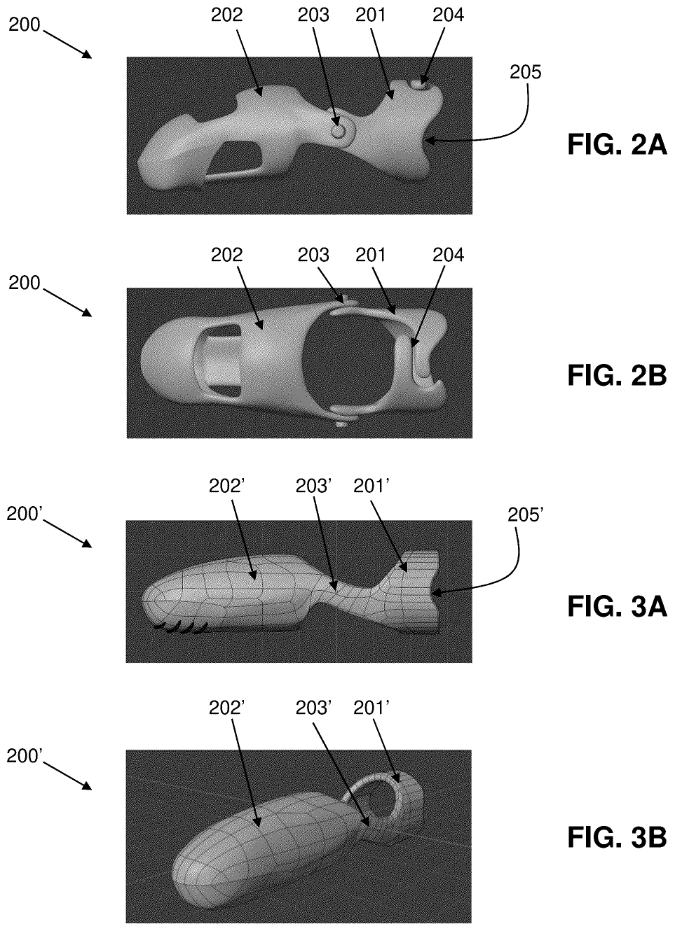

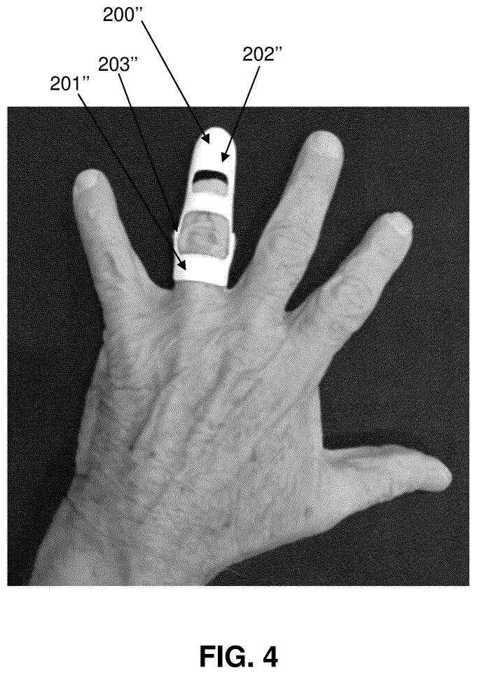

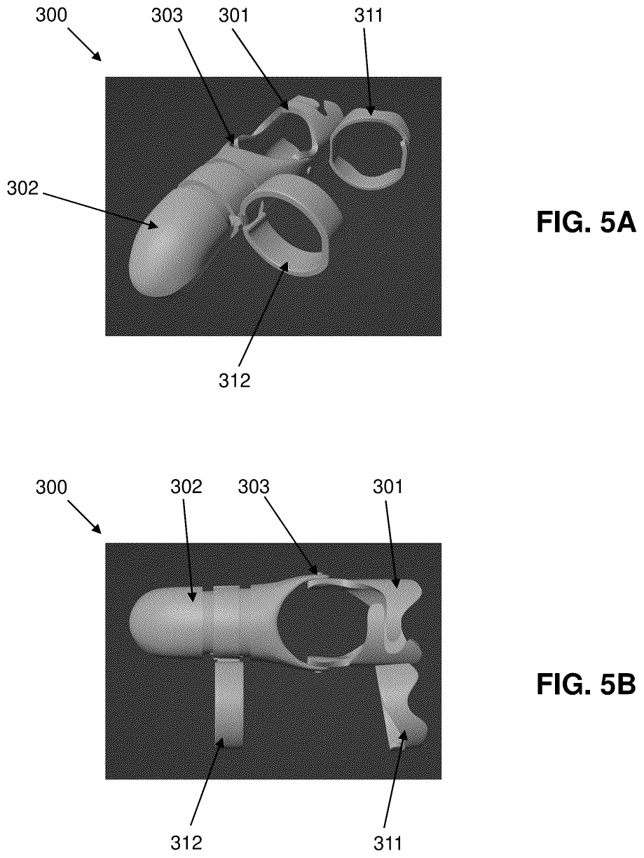

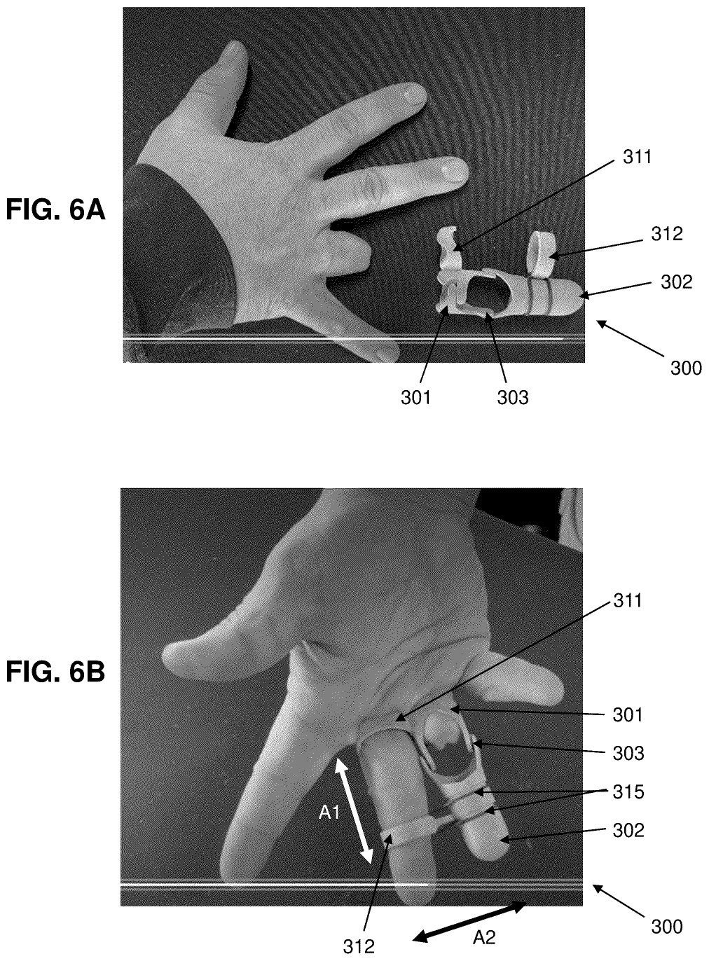

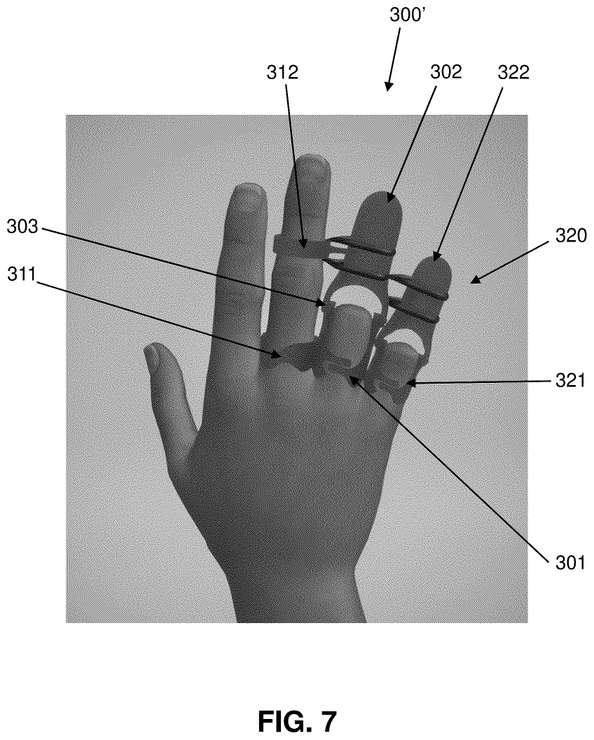

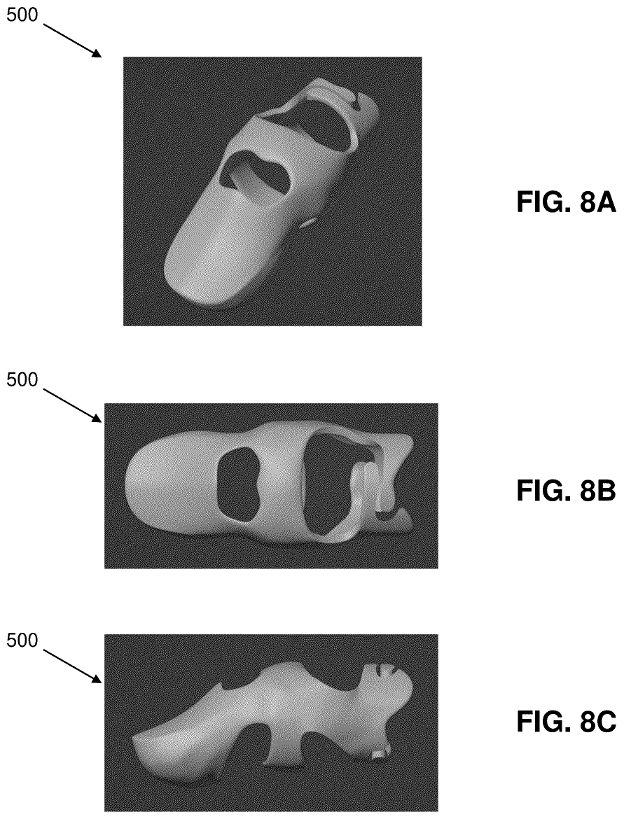

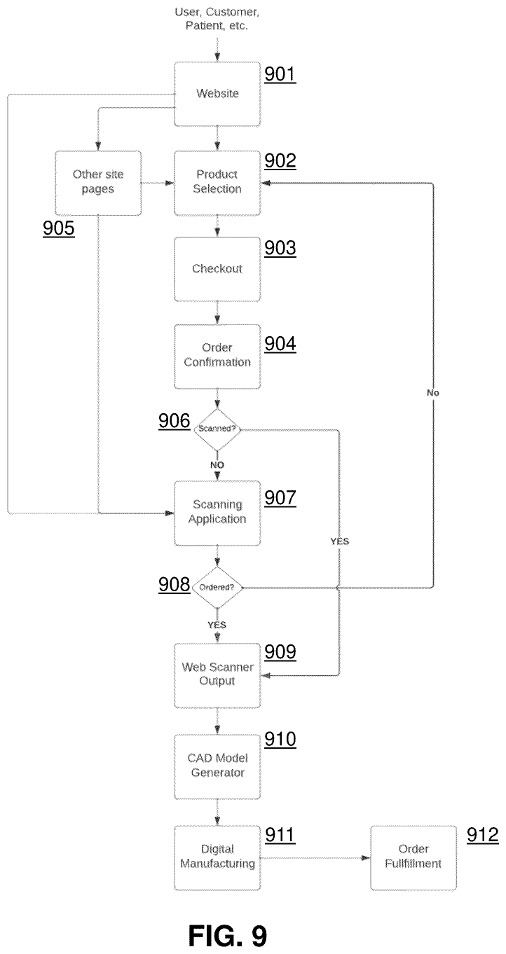

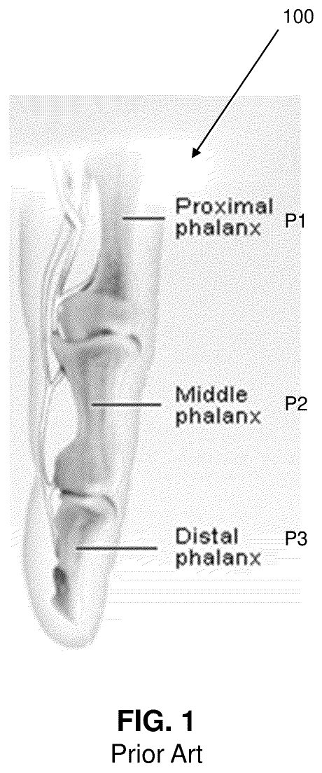

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

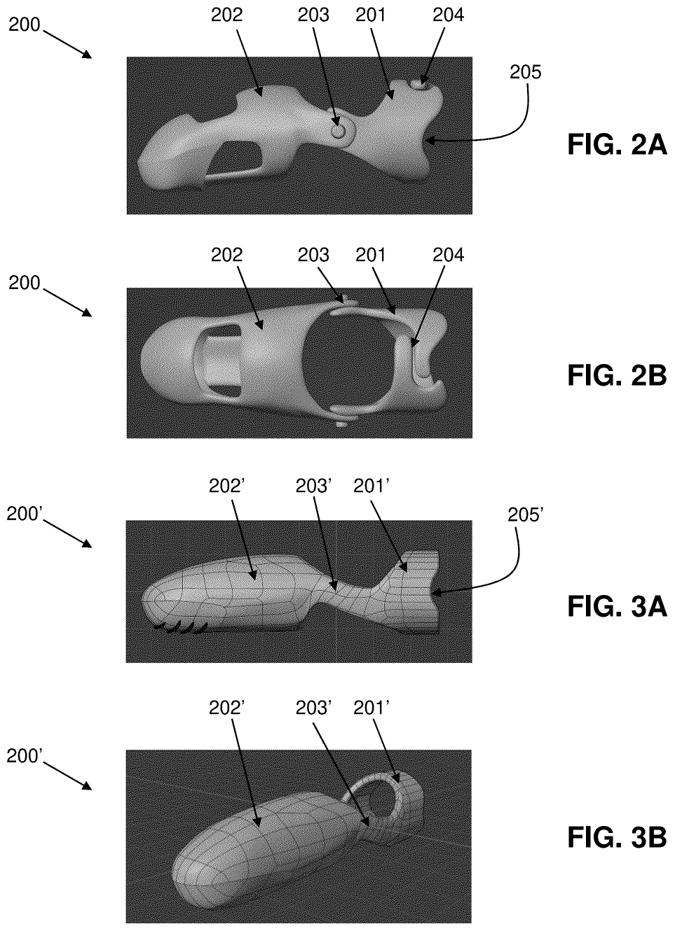

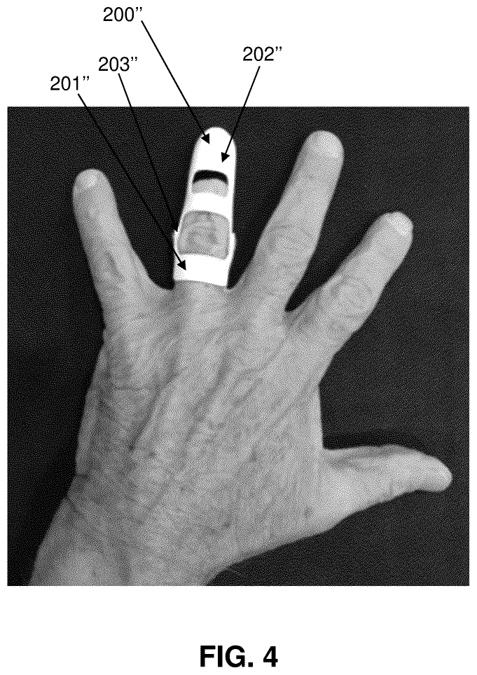

FIGS. 2A and 2B show a finger prosthetic 200 in accordance with aspects of the invention. The prosthetic 200 is designed for use with a finger that is missing the distal phalanx while still having the proximal phalanx and the middle phalanx. This design may be used for people that still have use of their middle phalanx and can bend the finger on their own. In embodiments, the prosthetic 200 includes a base piece 201 and a tip piece 202 connected by a hinge 203. The hinge 203 permits rotational movement of the tip piece 202 relative to the base piece 201. In embodiments, the base piece 201 is sized and shaped to be attached to the proximal phalanx portion of the user's finger and the tip piece 202 is sized and shaped to be attached to the middle phalanx portion of the user's finger, with the hinge 203 aligned with the joint between the proximal phalanx portion of the user's finger and the middle phalanx portion of the user's finger, e.g., as shown in FIG. 4. When the prosthetic 200 is attached to the user's amputated finger as shown in FIG. 4, the tip piece 202 may rotate relative to the base piece 201 in response to the user bending and straightening the finger. In this manner, the middle phalanx portion of the user's finger is used to drive the motion of the prosthetic 200.

In embodiments, the hinge 203 includes a pin hinge mechanism. For example, as shown in FIGS. 2A and 2B, the hinge 203 may include pins extending outward from the base piece 201, the pins being received in respective holes in the tip piece 202. Other hinges may be used. For example, the hinge may include function pin and slot, rotating or sliding joints, living hinges, etc. In one alternative example, the hinge may include pins extending inward from the tip piece 202, the pins being received in respective holes in the base piece 201. In another example in which the tip piece 202 is inside the base piece 201, the hinge may include pins extending outward from the tip piece 202, the pins being received in respective holes in the base piece 201. In yet another example, shown in FIGS. 3A and 3B, the hinge may include a living hinge.

In accordance with aspects of the invention, the hinge 203 is integrally formed with the base piece 201 and the tip piece 202. In the example of a pin hinge mechanism, the pins and holes are formed in the material of the base piece 201 and the tip piece 202, respectively, without any additional hardware being attached to the base piece 201 or the tip piece 202. In the example of a living hinge mechanism, the living hinge is formed integrally with material that connects the base piece 201 and the tip piece 202. In this manner, the prosthetic 200 can be fully additively manufactured with no extra hardware needed, with the hinges of the device manufactured into the device.

With continued reference to FIGS. 2A and 2B, the prosthetic 200 may include an expansion joint 204 in one or both of the base piece 201 and tip piece 202. In embodiments, the expansion joint 204 is a mechanism that provides for resiliently biased expansion of the base piece 201. In one example, the expansion joint 204 is configured to permit the diameter of the base piece 201 to be expanded outward (i.e., larger) when a sufficient force is applied to the base piece 201, and to resiliently bias the base piece back toward its original (unexpanded) shape when the force is removed. In this manner, the expansion joint 204 provides a mechanism for the base piece 201 to be expanded in diameter when being attached to the proximal phalanx portion of the user's amputated finger, and to snugly hold the base piece 201 on that portion of the finger due to the resilient biasing force causing the base piece 201 to contract onto the finger. In this manner, the expansion joint 204 provides a mechanism for the base piece 201 to flex and thereby accommodate a change in shape of the proximal phalanx portion, for example, when the user bends and straightens their finger. The prosthetic 200 shown in FIGS. 2A and 2B includes the expansion joint 204 in the base piece 201, but it is understood that an expansion joint could also be provided in the tip piece 202.

With continued reference to FIGS. 2A and 2B, the prosthetic 200 may include a web space 205 in the base piece 201. In embodiments, the web space 205 is a concave shaped portion of the base piece 201 that is designed to accommodate webbing between the amputated finger on which the prosthetic 200 is attached and neighboring fingers. In this manner, the web space 205 provides a more comfortable fit of the prosthetic 200 on the amputated finger.

In embodiments, the dimensions of the prosthetic 200 are determined using digital imaging and analysis. In a preferred embodiment, a scanning application installed on a user computing device (e.g., a smartphone, tablet computer, etc.), obtains images of the user's hand including the amputated finger. Dimensional data is determined from these images and used to generate a computer aided design (CAD) file with dimensions of the prosthetic 200 to match the measured dimensions of the user's amputated finger. In one example, the user places an object of known size (such as a quarter, dime, nickel, etc.) next to their hand when taking the digital images, and the scanning application uses the known size of the object and image analysis techniques to determine dimensions of the user's finger from the images. In embodiments, the CAD file is used to digitally manufacture the prosthetic 200, e.g., using 3D printing with high-grade nylon material that confers low cost while maintaining comfort and durability. In this manner, the prosthetic 200 is manufactured with a custom fit for the user, and at a minimal cost to the user. Aspects of the invention are not limited to 3D printing with nylon, and other conventional or later-developed manufacturing methods and/or materials may be used.

FIGS. 3A and 3B show a finger prosthetic 200′ in accordance with aspects of the invention. Similar to the prosthetic 200 of FIGS. 2A and 2B, the prosthetic 200′ of FIGS. 3A and 3B is designed for use with a finger that is missing the distal phalanx while still having the proximal phalanx and the middle phalanx. This design may be used for people that still have use of their middle phalanx and can bend the finger on their own. In embodiments, the prosthetic 200′ includes a base piece 201′ and a tip piece 202′ connected by a hinge 203′. The hinge 203′ permits rotational movement of the tip piece 202′ relative to the base piece 201′. In embodiments, the base piece 201′ is sized and shaped to be attached to the proximal phalanx portion of the user's finger and the tip piece 202′ is sized and shaped to be attached to the middle phalanx portion of the user's finger, with the hinge 203′ aligned with the joint between the proximal phalanx portion of the user's finger and the middle phalanx portion of the user's finger, e.g., as shown in FIG. 4. When the prosthetic 200′ is attached to the user's amputated finger (e.g., in the manner shown in FIG. 4), the tip piece 202′ may rotate relative to the base piece 201′ in response to the user bending and straightening the finger.

In the prosthetic 200′, the hinge 203′ comprises a living hinge. In embodiments, the living hinge is a thinned portion of the prosthetic 200′ between the base piece 201′ and the tip piece 202′ that allows more flexure of the prosthetic 200′ compared to the amount of flexure possible at other portions of the prosthetic 200′ not at the living hinge. Although not shown in FIGS. 3A and 3B, the prosthetic 200′ may include an expansion joint 204.

FIG. 4 shows a finger prosthetic 200″ installed on an amputated finger of a patient hand in accordance with aspects of the invention. The prosthetic 200″ includes a base piece 201″ and a tip piece 202″ connected by a hinge 203″ which function similar to those elements as described in prosthetic 200. Although not shown in FIG. 4, the prosthetic 200″ may include an expansion joint 204 and/or a web space 205 as in prosthetic 200.

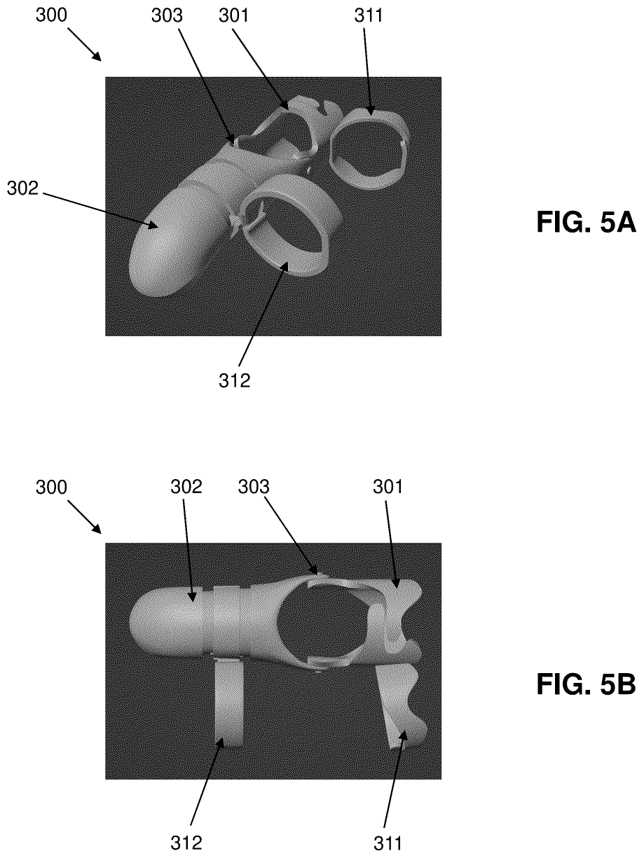

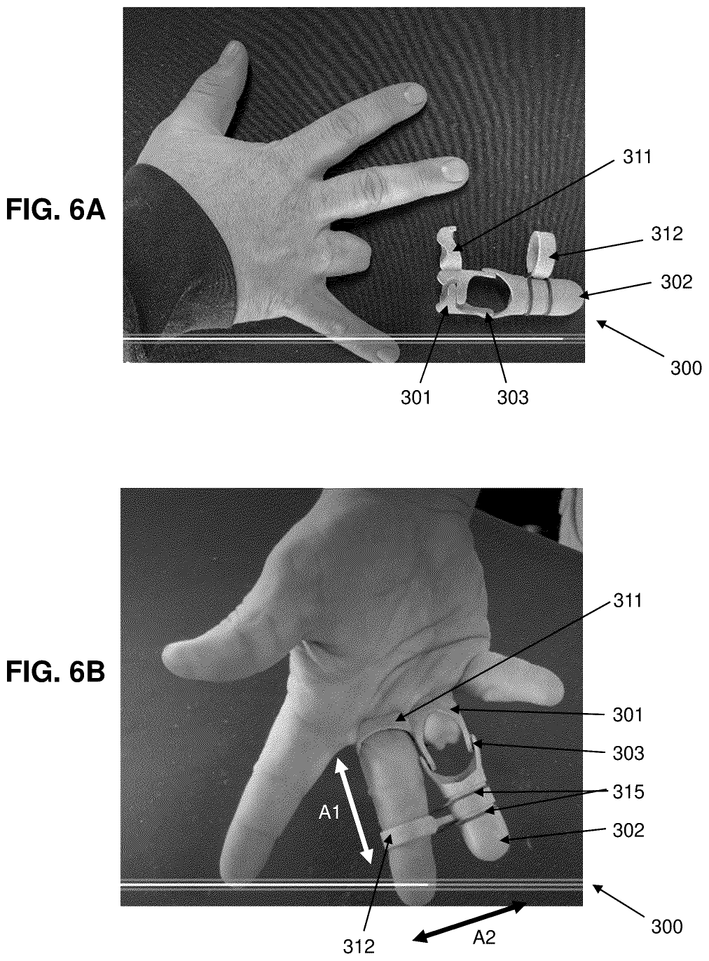

FIGS. 5A and 5B show a finger prosthetic 300 in accordance with aspects of the invention. The prosthetic 300 is designed for use with a finger that is missing the distal phalanx and the middle phalanx while still having the proximal phalanx, e.g., as illustrated in FIGS. 6A and 6B. In embodiments, the prosthetic 300 includes a base piece 301 and a tip piece 302 connected by a hinge 303. The hinge 303 permits rotational movement of the tip piece 302 relative to the base piece 301. In embodiments, the base piece 301 is sized and shaped to be attached to the proximal phalanx portion of the user's finger and the tip piece 302 is not directly connected to any portion of the finger, e.g., as shown in FIG. 6B.

With continued reference to FIGS. 5A and 5B, in embodiments the prosthetic 300 includes a base ring 311 connected to the base piece 301 and a tip ring 312 connected to the tip piece 302. In accordance with aspects of the invention, and as shown in FIG. 6B, the base ring 311 is configured to fit onto the proximal phalanx portion of the user's finger adjacent to the amputated finger, and the tip ring 312 is configured to fit onto the middle phalanx portion of the same finger adjacent to the amputated finger. When the prosthetic 300 is attached to the user's amputated finger and adjacent finger as shown in FIG. 6B, the tip piece 302 may rotate relative to the base piece 301 in response to the user bending and straightening the adjacent finger. In this manner, the user's finger that is adjacent to the amputated finger is used to drive the motion of the prosthetic 300.

In accordance with aspects of the invention, the connection between the tip ring 312 and the tip piece 302 permits limited movement of the tip ring 312 relative to the tip piece in a first direction and a second direction as indicated by arrows A1 and A2 in FIG. 6B. In one embodiment, the tip piece 302 includes a slot and the tip ring 312 includes a bar that is slidingly received the slot. In this embodiment, the slot and bar are sized and shaped relative to one another such that the bar can slide back and forth in the slot in the first direction A1 and such that the bar can slide into and out of the slot in the second direction A2. The bar and slot many be provided with structures (e.g., limit stops, etc.) that limit the amount of movement in both directions, and that keep the bar contained in the slot.

Motion in the second direction A2 permits spreading apart of the adjacent finger and the prosthetic 300 on the amputated finger, which spreading is desired during some hand movements. In embodiments, the prosthetic 300 includes a biasing element 315 that resiliently biases the tip ring 312 toward the tip piece 302 along the second direction A2. The biasing element 315 may comprise one or more elastic bands that encircle respective portions of the tip ring 312 toward the tip piece 302. In this manner, the user may use their strength to separate the tip ring 312 from the tip piece 302 along the second direction A2, and then rely on the biasing element to close the gap between the tip ring 312 and the tip piece 302.

In embodiments, and as shown in FIGS. 5A, 5B, 6A, and 6B, the base piece 301 and the base ring 311 each include an expansion joint that functions similar to expansion joint 204 shown in FIG. 2, and web spaces that function similar to web space 205 shown in FIG. 2. The prosthetic 300 shown in FIGS. 5A, 5B, 6A, and 6B includes a hinge 303 similar to the hinge 203 shown in FIG. 2; however, other types of hinge may be used as described herein. The base piece 301, base ring 311, tip piece 302, and tip ring 312 may be manufactured in the same manner as described with respect to prosthetic 200, e.g., using image scanning and analysis and 3D printing.

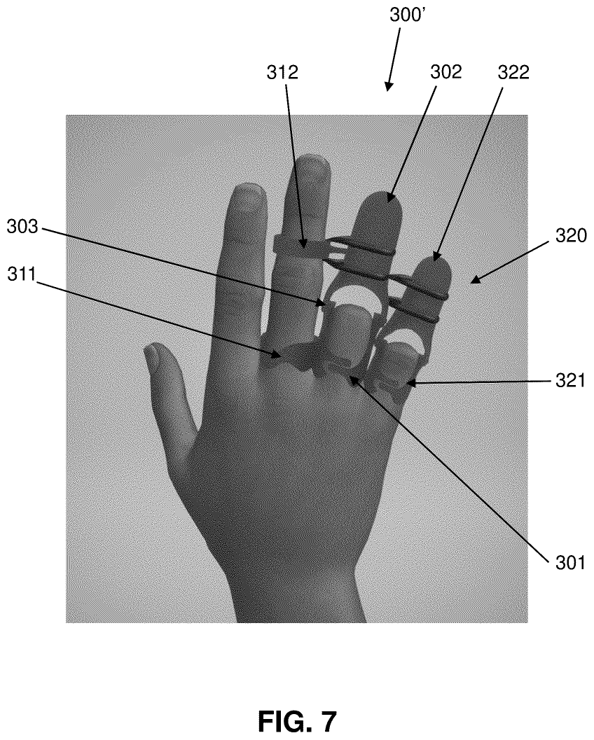

FIG. 7 shows a finger prosthetic 300′ in accordance with aspects of the invention. The prosthetic 300′ includes the same elements 301, 302, 303, 311, 312, and 315 as prosthetic 300, and additionally includes cascaded prosthetic finger 320 that includes a base piece 321 and tip piece 322 that are hinged to one another and that are connected to the base piece 301 and tip piece 302, respectively. Using the prosthetic 300′, and single finger 400 can be used to drive prosthetic tips for two amputated fingers.

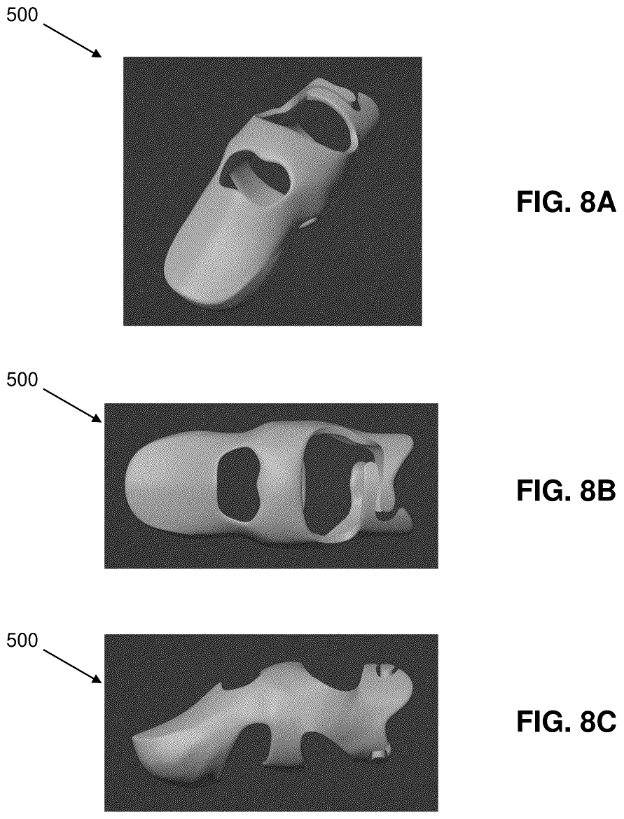

FIGS. 8A, 8B, and 8C show a finger prosthetic 500 in accordance with aspects of the invention. The prosthetic 500 does not include a hinge and can be used, for example, on a partially amputated thumb. The prosthetic 500 includes a base portion that fits onto a remaining portion of the amputated thumb and a tip portion that take the place of the missing portion of the thumb. The prosthetic 500 may include an expansion joint and/or web spaces that function as described herein. The prosthetic 500 may be manufactured in the same manner as described with respect to prosthetic 200, e.g., using image scanning and analysis and 3D printing.

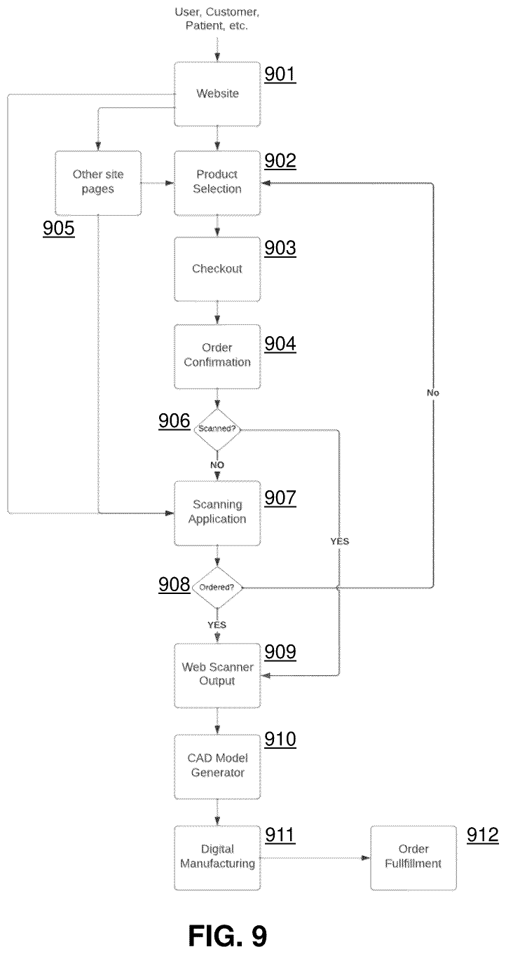

FIG. 9 shows a flowchart of a method in accordance with aspects of the invention.

Step 901 includes a user engagement that allows or directs users to navigate to a proceeding step. In embodiments, the user engagement is a web page. In embodiments, the user can navigate to steps 902, 905 or 907.

Step 902 includes a user engaging with a method to select a product. In embodiments, the user can make selections to choose the product and product features they would like to proceed with. In embodiments, the user can proceed to step 903 after sections have been made.

Step 903 includes a user engaging a checkout method to process payments and collect user information. In embodiments, the user provides personal information and payment information. In embodiments, the user submits the order via a submission button.

Step 904 includes a user engaging a step that confirms the order and directs the user to the following step. In embodiments, the user is shown a confirmation window or is sent a confirmation email that includes links to the following step.

Step 905 includes a user engaging a web page that provides additional information. In embodiments the user can continue webpage navigation, continue to steps 907 or 905.

Step 906 includes a decision step. In embodiments, if the user has already scanned the hand, they are directed to step 909. In embodiments, if the user has not scanned the hand, they are directed to step 907.

Step 907 includes a user engaging with the scanning application. In embodiments, the user is prompted to scan each hand using the scanning application process defined earlier in the specification. In embodiments, the image data includes images of the user's hand that has the amputated finger. In embodiments, at one of the images of the user's hand include an object of known size, such as a coin (e.g., quarter, dime, nickel, etc.). In embodiments, the scanning application provides the data required to continue in the process.

Step 908 includes a decision step. In embodiments, if the user has already proceeded through steps 902, 903 and 904 they are directed to step 909. In embodiments, if the user has not completed steps 902, 903 and 904 they are directed to step 902. In embodiments, step 908 can make a decision for step 902 and send the user to step 903.

Step 909 includes obtaining a web scanner output. In embodiments, a computer system of the supplier receives image data obtained by the user computing device (e.g., smartphone, tablet computer, etc.) via network communication. In embodiments, the image data include images of the user's hand that has the amputated finger. In embodiments, at one of the images of the user's hand include an object of known size, such as a coin (e.g., quarter, dime, nickel, etc.).

Step 910 includes generating a CAD model based on the web scanner output from step 909. In embodiments, the computer system of the supplier uses the known size of the object and image analysis techniques (e.g., edge detection, etc.) to determine dimensions of plural portions of the user's hand that has the amputated finger. In embodiments, when generating a prosthetic 200, the system uses these techniques to determine at least a diameter of the proximal phalanx portion of the amputated finger for the base piece 201. In embodiments, when generating a prosthetic 300, the system uses these techniques to determine at least a diameter of the proximal phalanx portion of the amputated finger for the base piece 301, a diameter of the proximal phalanx portion of the adjacent finger for the base ring 311, and a diameter of the middle phalanx portion of the adjacent finger for the tip ring 312. In an additional embodiment, the image data of step 909 includes an image of the user's other hand that does not have the amputated finger. In this embodiment, the system determines a length of the tip piece (202 or 302) based on a length of a whole finger on the other hand, the whole finger corresponding to the amputated finger. For example, if the user's third finger on their right hand is the amputated finger, then the system uses the length of the third finger on the left hand to determine the length of the tip piece. In embodiments, the system generates a CAD model of the prosthetic using the determined dimensions.

Step 911 includes manufacturing the prosthetic using the CAD model from step 910, In embodiments, the computer system of the supplier 3D prints the prosthetic using the CAD model from step 910.

Step 912 includes fulfilling the order to the customer. In embodiments, the supplier mails the prosthetic that was generated at step 911 to the customer.

Additional aspects of the invention include manufacturing and/or using a finger prosthetic (200 or 200′ or 200″ or 300 or 500) as described herein. Even further aspects of the invention include providing instructions for using a finger prosthetic (200 or 200′ or 200″ or 300 or 500) as described herein. The instructions may be at least one of printed and video.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

What is claimed:

1. A prosthetic device for use with an amputated finger that is missing a distal phalanx and some or all of a middle phalanx, the device comprising:
a base piece configured to fit on a proximal phalanx portion of the amputated finger;
a tip piece pivotally connected to the base piece by a hinge that is integrally formed with the tip piece and the base piece;
a base ring configured to fit on a proximal phalanx portion of a finger adjacent to the amputated finger, wherein the base ring is connected to the base piece; and
a tip ring configured to fit on a middle phalanx portion of the finger adjacent to the amputated finger, wherein the tip ring is connected to the tip piece.

2. The device of claim 1, wherein the connection between the tip ring and the tip piece permits translational movement of the tip ring relative to the tip piece in two orthogonal directions.

3. The device of claim 2, wherein a first of the two orthogonal directions is along a longitudinal direction of the tip piece and a second of the two orthogonal directions is transverse to the longitudinal direction of the tip piece.

4. The device of claim 3, further comprising a biasing element that resiliently biases the tip ring toward the tip piece along the second of the two orthogonal directions.

5. The device of claim 1, wherein the hinge comprises:
pins integrally formed with the base piece; and
holes integrally formed in the tip piece, wherein respective ones of the pins are received in respective ones of the holes.

6. The device of claim 1, wherein the hinge comprises:
pins integrally formed in a material of the tip piece; and
holes integrally formed in a material of the base piece, wherein respective ones of the pins are received in respective ones of the holes.

7. The device of claim 1, wherein the hinge comprises a living hinge integrally formed with both the base piece and the tip piece.

8. The device of claim 1, wherein the base piece comprises an expansion joint.

9. The device of claim 1, wherein the base piece comprises a web space.

10. The device of claim 9, wherein the web space comprises a concave shaped portion of the base piece that, when the device is installed on the amputated finger, accommodates webbing between the amputated finger on which the prosthetic device is attached and a neighboring finger.

11. The device of claim 9, wherein:
the base piece comprises an expansion joint that provides for resiliently biased expansion of a diameter of the base piece; and
the web space comprises a concave shaped portion of the base piece that, when the device is installed on the amputated finger, accommodates webbing between the amputated finger on which the prosthetic device is attached and a neighboring finger.

12. The device of claim 1, wherein, when the device is installed on the amputated finger, the base piece encircles the proximal phalanx portion of the amputated finger.

13. The device of claim 1, wherein, when the device is installed on the amputated finger, the tip piece is spaced apart from the amputated finger.

14. The device of claim 1, wherein, when the device is installed on the amputated finger:
the base piece encircles the proximal phalanx portion of the amputated finger; and
the tip piece is spaced apart from the amputated finger.

15. A method of manufacturing a prosthetic device for use with an amputated finger, the method comprising:
receiving at least one digital image that includes an image of the amputated finger and an object of known size;
determining at least one dimension of the amputated finger using the at least one digital image;
generating a CAD file for the prosthetic device based on the determined at least one dimension; and
digitally manufacturing the prosthetic device using the CAD file.

16. The method of claim 15, wherein the digitally manufacturing the prosthetic device comprises 3D printing the prosthetic device using the CAD file.

17. The method of claim 15, wherein the prosthetic device comprises:
a base piece configured to fit on a proximal phalanx portion of the amputated finger; and
a tip piece configured to fit on a middle phalanx portion of the amputated finger,
wherein the tip piece is pivotally connected to the base piece by a hinge that is integrally formed with the tip piece and the base piece.

18. The method of claim 15, wherein the prosthetic device comprises:
a base piece configured to fit on a proximal phalanx portion of the amputated finger;
a tip piece pivotally connected to the base piece by a hinge that is integrally formed with the tip piece and the base piece;
a base ring configured to fit on a proximal phalanx portion of a finger adjacent to the amputated finger, wherein the base ring is connected to the base piece; and
a tip ring configured to fit on a middle phalanx portion of the finger adjacent to the amputated finger, wherein the tip ring is connected to the tip piece.
